(19) United States
(12) Patent Application Publication
(10) Pub. No.: US 2025/0366722 A1
(43) Pub. Date: Dec. 4, 2025

(54) TECHNIQUES FOR PHOTOPLETHYSMOGRAM ANALYSIS BASED ON ATTRACTOR RECONSTRUCTION

(71) Applicant: Oura Health Oy, Oulu (FI)

(72) Inventor: Antti Aleksi Rantanen, Oulu (FI)

(21) Appl. No.: 19/220,619

(22) Filed: May 28, 2025

Related U.S. Application Data
(60) Provisional application No. 63/653,115, filed on May 29, 2024.

(51) Int. Cl. A61B 5/0205; A61B 5/00; A61B 5/021; A61B 5/024; A61B 5/1455; G16H 40/63; G16H 50/70

(52) U.S. Cl. CPC A61B 5/0205; A61B 5/02108; A61B 5/02405; A61B 5/02416; A61B 5/14551; A61B 5/6802; A61B 5/7221; A61B 5/7239; A61B 5/742; A61B 5/7475; G16H 40/63; G16H 50/70; A61B 2560/0462

(57) ABSTRACT

Methods, systems, and devices for photoplethysmogram (PPG) analysis are described. Techniques described herein may enable a system to convert a PPG signal from a time-domain signal into an attractor reconstruction representation in three-dimensional (3D) space. Visualizations of such 3D attractor reconstruction projections may be displayed to a user via a smart device. Further, the system may use machine learning models to identify morphological features within 3D attractor reconstruction projections to perform physiological measurements and determine health-related insights for the user. For example, a smart device may input time-domain PPG signals and 3D attractor reconstruction projections into machine learning models that are configured to identify morphological features within the time-domain PPG signals and attractor reconstruction projections. The smart device may perform various physiological measurements, such as cardiovascular age, blood pressure, heart rate variability, and the like.

### CROSS-REFERENCE

The present application for patent claims the benefit of U.S. Provisional Patent Application No. 63/653,115 by Rantanen, entitled “TECHNIQUES FOR PHOTOPLETHYSMOGRAM ANALYSIS BASED ON ATTRACTOR RECONSTRUCTION” and filed May 29, 2024, which is assigned to the assignee hereof and is hereby expressly incorporated by reference herein in its entirety.

### FIELD OF TECHNOLOGY

The following relates to wearable devices and data processing, including techniques for photoplethysmogram (PPG) analysis based on attractor reconstruction.

### BACKGROUND

Some wearable devices may be configured to collect data from users associated with photoplethysmogram (PPG) measurements. The wearable devices may be configured to analyze the PPG measurements to determine health information about the user.

### BRIEF DESCRIPTION OF THE DRAWINGS

FIG. 1 illustrates an example of a system that supports techniques for photoplethysmogram (PPG) analysis based on attractor reconstruction in accordance with aspects of the present disclosure.

FIG. 2 illustrates an example of a system that supports techniques for PPG analysis based on attractor reconstruction in accordance with aspects of the present disclosure.

FIG. 3 shows an example of a data diagram that supports techniques for PPG analysis based on attractor reconstruction in accordance with aspects of the present disclosure.

FIG. 4 shows an example of a graphical user interface (GUI) that supports techniques for PPG analysis based on attractor reconstruction in accordance with aspects of the present disclosure.

FIG. 5 shows a flowchart illustrating methods that support techniques for PPG analysis based on attractor reconstruction in accordance with aspects of the present disclosure.

### DETAILED DESCRIPTION

Wearable devices can be configured to collect physiological data from users to provide users with more information regarding their overall health, including photoplethysmogram (PPG) data. The PPG data may include data related to heart health of the user (e.g., cardiovascular age, heart rate, interbeat interval (IBI), blood pressure, and the like). In some examples, a system may analyze and display PPG data for the user as a function of time (e.g., based on time-domain representations of PPG signals). However, such time-domain representations of PPG data may be unintuitive for users to interpret. Moreover, depending on the quality of the PPG data, it may be difficult to identify features (e.g., regularity of blood pressure, regularity of heart rate, and the like) within such time-domain PPG signals. Thus, a system to analyze the PPG data (e.g., machine learning data analysis systems) may be unsuccessful in deriving health-related insights from the PPG data. Further, identifying or extracting features from such time-domain PPG signals may be computationally expensive, and may use extensive memory and processing capabilities.

Accordingly, techniques described herein may enable a system (e.g., a wearable device, a user device, servers, etc.) to convert a PPG signal from a time-domain signal into an attractor reconstruction (e.g., a Symmetric Projection Attractor Reconstruction (SPAR)) representation in two-dimensional (2D) and/or three-dimensional (3D) space. Visualizations of such attractor reconstruction projections may be displayed to a user via a smart device, and may provide a more intuitive format for visualizing the user's physiological data as compared to a time-domain representation. Further, machine learning models may be used to identify morphological features within reconstructed PPG projections (in addition to or instead of morphological features within time-domain PPG signals) to perform physiological measurements. As such, techniques described herein may be used to generate more accurate health-related insights. For example, time-domain PPG signals and reconstructed PPG projections may be input into machine learning models that are configured to identify morphological features within the PPG signals and reconstructed PPG projections to perform various physiological measurements, such as cardiovascular age, blood pressure, heart rate variability (HRV), and the like.

In some implementations, morphological features of collected PPG data, which may be determined based on the reconstructed PPG projections, may be used to adjust or modify operational parameters (e.g., light-emitting diode (LED) intensity, LED burn time, wavelengths of light, of the wearable device used to acquire subsequent PPG data. By modifying operational parameters of the wearable device based on determined morphological features, aspects of the present disclosure may improve the quality and reliability of subsequent physiological data collected by the wearable device.

Aspects of the disclosure are initially described in the context of systems supporting physiological data collection from users via wearable devices. Aspects of the disclosure are further illustrated by and described with reference to data diagrams, GUIs, apparatus diagrams, system diagrams, and flowcharts that relate to techniques for PPG analysis based on attractor reconstruction.

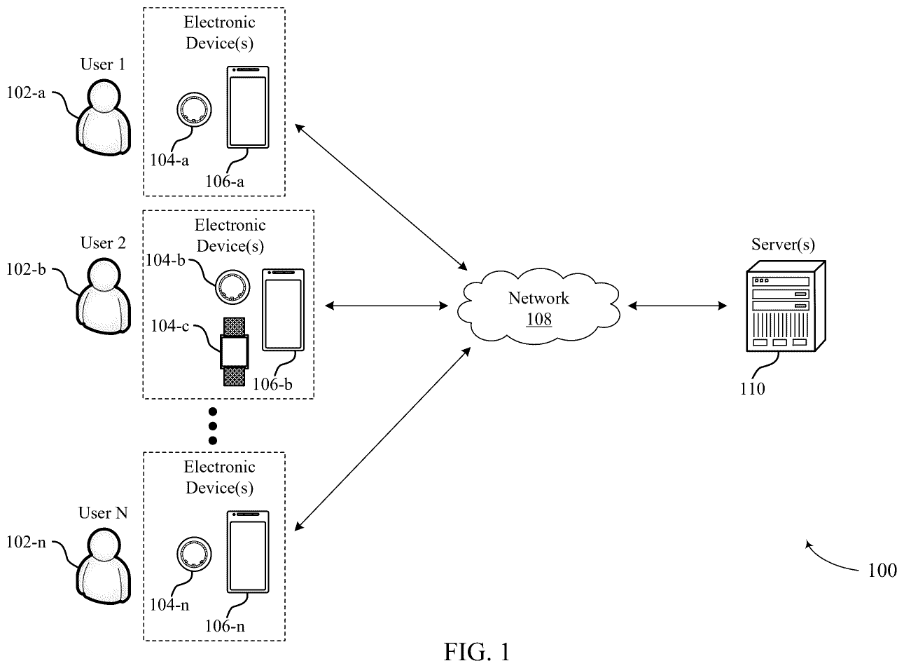

FIG. 1 illustrates an example of a system 100 that supports techniques for PPG analysis based on attractor reconstruction in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, HRV, actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols. Moreover, in some cases, the wearable device 104 and the user device 106 may be included within (or make up) the same device. For example, in some cases, the wearable device 104 may be configured to execute an application associated with the wearable device 104, and may be configured to display data via a GUI.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 (e.g., a wearable device 104, a user device 106) may support techniques to converting a PPG signal from a time-domain PPG signal into an attractor reconstruction (e.g., a SPAR) representation in 2D and/or 3D space. Visualizations of such attractor reconstruction projections (e.g., reconstructed PPG projections) may be displayed to a user via the user device 106, and may provide a more intuitive format for visualizing physiological data of the user as compared to a time-domain representation. Further, machine learning models may be used to identify morphological features within reconstructed PPG projections (in addition to or instead of morphological features within time-domain PPG signals) to perform physiological measurements. As such, techniques described herein may be used to generate more accurate health-related insights. For example, the user device 106 may input time-domain PPG signals and reconstructed PPG projections (e.g., 2D/3D SPAR projections) into machine learning models that are configured to identify morphological features within the PPG signals and reconstructed PPG projections to perform various physiological measurements, such as cardiovascular age, blood pressure, HRV, and the like.

It should be appreciated by a person skilled in the art that one or more aspects of the disclosure may be implemented in a system 100 to additionally, or alternatively, solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

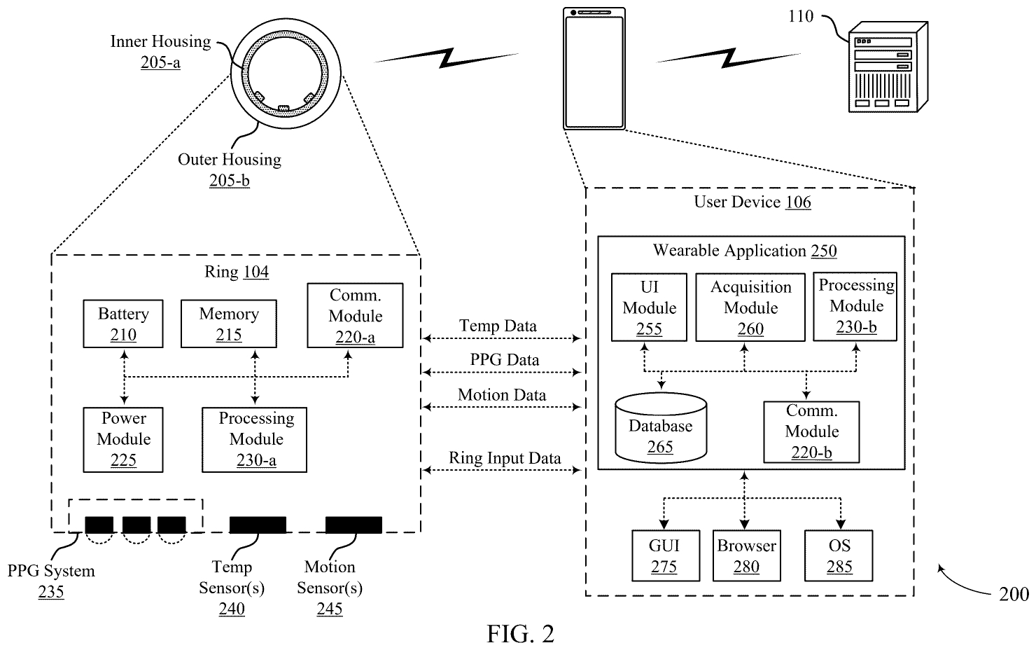

FIG. 2 illustrates an example of a system 200 that supports techniques for PPG analysis based on attractor reconstruction in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, PPG data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an IBI. The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BMI160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

In some cases, the wearable device 104 and the user device 106 may be included within (or make up) the same device. For example, in some cases, the wearable device 104 may be configured to execute the wearable application 250, and may be configured to display data via the GUI 275.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, data collected by the wearable device 204, and/or analyses performed by the wearable device 204, the user device 206, and/or the servers 210, may be used to adjust operational parameters of the wearable device 204. For example, based on a determined heart rate of the user and/or a determined activity state of the user, the wearable device 204 may adjust a sampling rate for measuring the user's heart rate, and/or may activate or deactivate certain sensors and/or physiological measurements (e.g., deactivate SpO2 measurements when the user is engaged in physical activity, or otherwise exhibits an activity/movement level above some threshold). By way of another example, the user device 206 and/or the servers 210 may calculate a Readiness Score for the user, and may deactivate or disable activity measurements performed by the wearable device 204 in cases where the Readiness Score is below some threshold (in order to reduce power consumption and conserve battery at the wearable device 204, and/or to disincentivize the user from performing rigorous activity when their Readiness Score is below the threshold value). In this regard, any measurements, calculations, and/or analyses performed by the various devices within the system 200 (e.g., wearable device 204, user device 206, servers 210) may be used by the system 200 to control and/or adjust the operational parameters of the wearable device 204.

Operational parameters that may be controlled/adjusted at the wearable device 204 based on collected data and/or analyses performed by the system 200 may include, but are not limited to, a periodicity/frequency that measurements are performed (e.g., sampling rate), a power level or intensity of LEDs, algorithms used to analyze data at the wearable device 204, what types of measurements are performed (e.g., enabling/disabling specific sensors or types of measurements), a periodicity or frequency that the wearable device 204 transmits data to the user device 206, or any combination thereof. Adjusting operational parameters of the wearable device 204 based on collected data and/or analyses performed by the system 200 may reduce power consumption and improve battery performance at the wearable device 204, and may lead to higher quality data collected by the wearable device 204, thereby enabling the system 200 to perform more accurate and reliable analyses/diagnoses of the user's physiological parameters, and leading to better guidance and insights that may enable the user to improve their overall health.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the system 200 (e.g., a wearable device 104, a user device 106) may support techniques for converting a PPG signal from a time-domain PPG signal into an attractor reconstruction (e.g., a SPAR) representation in 2D and/or 3D space. Visualizations of such attractor reconstruction projections (e.g., reconstructed PPG projections, SPAR projections) may be displayed to a user via the user device 106, and may provide a more intuitive format for visualizing physiological data of the user as compared to a time-domain representation. The user device 106 may display the attractor reconstruction representation of the PPG signals (e.g., as a 2D image, a 3D image, and/or an animation) to the user via the GUI. In some examples, the user device may display one or more health-related insights (e.g., insights determined based on the attractor reconstruction representation of the PPG signals) to the user via the GUI 275.

In some examples, the system 200 may use one or more machine learning models to identify morphological features within attractor reconstruction projections (in addition to or instead of morphological features within time-domain PPG signals). For example, the user device 106 may include one or more machine learning models configured to identify physiological features using one or more both of a time-domain PPG signal and an attractor reconstruction projection of the PPG signal. The one or more machine learning models may be trained via training datasets including ground-truth labels related to whether one or more features of training PPG signals (e.g., a shape, regularity, overlap, and so on of the training PPG signals) are indicative of certain health conditions of the user (e.g., blood pressures, cardiovascular age, heart rate, HRV, and the like).

As such, techniques described herein may be used to generate more accurate health-related insights. For example, the machine learning models may identify morphological features within the PPG signals and attractor reconstruction projections. The user device 106 may therefore determine health insights for the user, such as cardiovascular age, blood pressure, HRV, and the like. The user device 106 may therefore display the health insights via the GUI 275.

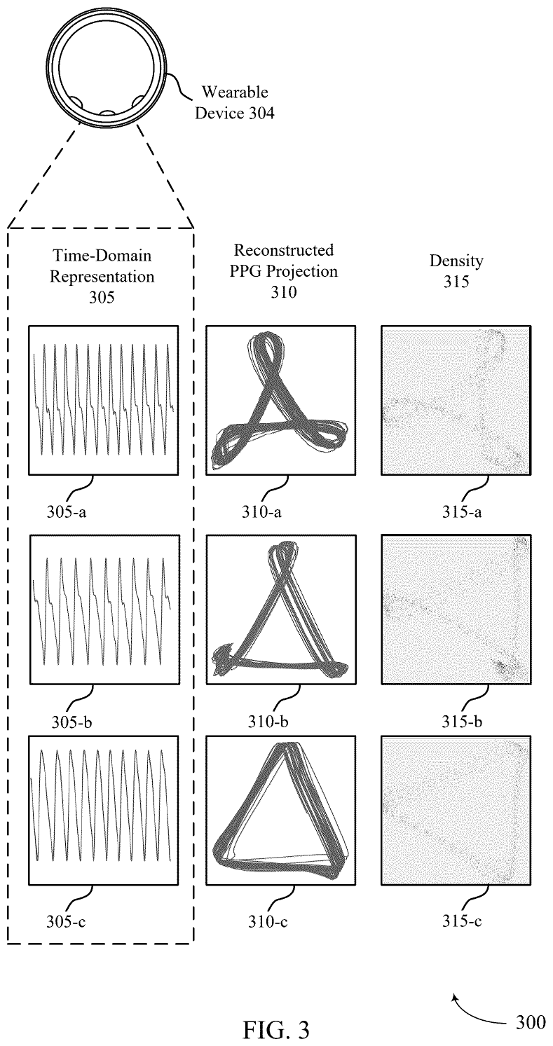

FIG. 3 shows an example of a data diagram 300 that supports techniques for PPG analysis based on attractor reconstruction in accordance with aspects of the present disclosure. The data diagram 300 may implement, or may be implemented by, aspects of the system 100 and the system 200. For example, the data diagram 300 may be used by a wearable device 304 or a user device 106, which may be examples of wearable devices 104 or user devices 106 as described with reference to FIG. 1.

In some examples, a wearable device 304 (e.g., a wearable ring device, a wrist-worn wearable device) may acquire physiological data from a user, including PPG data. The wearable device 304 may collect the PPG data using one or more sensors of the wearable device, such as one or more light-emitting components (e.g., LEDs) and one or more light-receiving components (e.g., photodiodes). The collected PPG data may include time-domain representations 305 (e.g., a time-domain signal, a one-dimensional (1D) signal varying over time) of cardiovascular data associated with the user, including changes in a blood volume of the user over time. For example, FIG. 3 illustrates three separate time-domain representations 305-a, 305-b, 305-c illustrating 1D PPG signals that exhibit different characteristics or morphological features. The different time-domain representations 305 may be collected from different users, or may represent PPG data collected from the same user during different time intervals and/or using different sensors or wavelengths. For example, the first time-domain representation 305-a may represent PPG data collected via the wearable device 304 using green light, the second time-domain representation 305-b may represent PPG data collected via the wearable device 304 using red light, and the third time-domain representation 305-c may represent PPG data collected via the wearable device 304 using IR light.

In some examples, the time-domain representation 305 of the PPG data may be relatively less informative than one or more other data representations. For example, a user may be unaware of how to interpret the time-domain representation 305, and a system (e.g., including a user device 106) may generate relatively less accurate health insights (e.g., cardiovascular health information, such as cardiovascular age) using the time-domain representation 305. Accordingly, the user device 106 and/or the wearable device 304 may perform an attractor reconstruction procedure to project the PPG data from the time-domain representation(s) 305 to a reconstructed PPG projection(s) 310 (e.g., a SPAR projection). The reconstructed PPG projections 310 may be a 2D visualization in 2D space or a 3D visualization in 3D space. For example, the system may perform an attractor reconstruction procedure to project the PPG data from the time-domain representation 305-a, 305-b, and 305-c to the reconstructed PPG projections 310-a, 310-b, and 310-c, respectively.

For instance, the system may generate an N dimensional reconstruction of time-domain signal (e.g., a 2D representation, a 3D representation) using a vector of time delay coordinates, such as [x(t), x(t−τ), x(t−2τ), . . . x(t−(N−1)t)], where τ>0 is a fixed delay (e.g., one third of an average PPG cycle length of the user). In some examples, to generate a 3D representation of the time-domain signal (e.g., in an (x, y, z) coordinate plane), a variable y(t) may be defined as y(t)=x(t−τ) and a variable z(t) may be defined as z(t)=x(t−2τ). Accordingly, the system may plot the time-domain representation 305 in a 3D space.

To generate a 2D projection of the 3D representation of the PPG data, the system may define a coordinate plane that is perpendicular to a vector [1, 1, 1]. For example, a variable u may be defined as

$$u = \frac{1}{3} ( x + y + z ) ,$$

a variable v may be defined as

$$v = \frac{1}{\sqrt{6}} ( x + y - 2 z ) ,$$

and a variable w may be defined as

$$w = \frac{1}{\sqrt{2}} ( x - y ) . A ( v , w )$$

coordinate plane may be a plane that is perpendicular to a vector [1, 1, 1]. By projecting the 3D representation into the (v, w) coordinate plane, the system may generate the reconstructed PPG projection 310. In some examples, a “loop” of the reconstructed PPG projection 310 (e.g., from a first point of the reconstructed PPG projection 310 clockwise around the triangular shape of the reconstructed PPG projection 310 and back to a point that is relatively close to or within a threshold (v, w) of the first point) may correspond to one PPG cycle (e.g., one cycle of PPG data, three times the fixed delay τ>0).

In some examples, the wearable device 304 may collect the physiological data (e.g., the time-domain representations 305 of the PPG data) using light from multiple wavelengths (e.g., red light, green light, IR light, and so on). For example, the wearable device may collect a first set of PPG data using a first wavelength of light and a second set of PPG data using a second wavelength of light. In such examples, the system may use two time-domain representations 305 (e.g., a time-domain representation 305 associated with each set of PPG data) to generate two reconstructed PPG projections 310 (e.g., via the attractor reconstruction procedure). For instance, the first reconstructed PPG projection 310-a may be associated with PPG data collected using green light, the second reconstructed PPG projection 310-b may be associated with PPG data collected using red light, and the third reconstructed PPG projection 310-c may be associated with PPG data collected using IR light.

In some examples, the system may determine a data quality metric associated with the PPG data (e.g., a received signal strength, a quality of the time-domain representation 305). The system may perform the attractor reconstruction procedure based on the data quality meeting a threshold data quality. For example, the system may refrain from performing the attractor reconstruction procedure if the PPG data does not meet the data quality threshold. In some examples, the system may perform one or more operations (e.g., adjusting operational parameters of the wearable device 304, such as enabling or disabling sensors, adjusting LED intensity, and the like) based on the data failing to satisfy the quality threshold. In other words, if physiological data collected by the wearable device 304 does not satisfy the quality threshold, the system may adjust operational parameters used by the wearable device 304 in order to improve the quality/reliability of subsequently-collected physiological data (e.g., so that subsequently-collected physiological data satisfies the quality threshold and may be used for the attractor reconstruction procedure).

In some aspects, the system may perform one or more operations to determine physiological characteristics of the user based on the reconstructed PPG projection(s) 310. For example, the system may input the PPG data (e.g., the time-domain representation(s) 305) and/or the reconstructed PPG projection(s) 310 into an algorithm, such as one or more machine learning models, neural networks, etc. The one or more machine learning models may be trained to identify the physiological characteristics associated with the user based on a first set of morphological features of the PPG data and/or a second set of morphological features of the reconstructed PPG projection(s) 310. The reconstructed PPG projection(s) 310 may be associated with a relatively improved success of the one or more machine learning models in determining morphological PPG features and/or physical conditions of the user as compared to the time-domain representation(s) 305. In other words, it has been found that the PPG projections 310 may enable more accurate and reliable determination of morphological PPG features and/or physical conditions (as compared to the time-domain representations 305).

For example, in some cases, the first time-domain representation 305-a of the PPG data may be input into a machine learning model, along with the first reconstructed PPG projection 310-a and “known” physiological metrics corresponding to (e.g., measured from) the time-domain representation 305-a. For instance, the system may determine that the first time-domain representation 305-a is associated with a certain heart rate, blood pressure, cardiovascular age, etc. In this regard, the machine learning model may be configured to “learn” that morphological features of the reconstructed PPG projection 310-a (and morphological features of the time-domain representation 305) are indicative of or otherwise associated with the respective heart rate, blood pressure, cardiovascular age, etc.

In some examples (e.g., if the wearable device 304 collects two sets of PPG data using light of different wavelengths and generates two reconstructed PPG projections 310), the system may input an additional reconstructed PPG projection 310 into the one or more machine learning models. The one or more machine learning models may be trained to identify the physiological characteristics based on a third set of morphological features associated with an additional reconstructed PPG projection 310. That is, the machine learning model may be configured to identify that morphological features of the respective reconstructed PPG projections 310 are associated with certain physiological characteristics. Further, the machine learning model may be configured to identify or “learn” relationships between morphological features of multiple different reconstructed PPG projections 310, and how such relationships or combinations of morphological features may be indicative of certain physiological characteristics.

In some examples, the one or more machine learning models may be trained based on a training dataset. The training dataset may include sets of PPG data (e.g., time domain representations 305, reconstructed PPG projections 310) collected from one or more users (e.g., one or more users associated with a plurality of demographic characteristics, such as ages, cardiovascular ages, activity levels, and the like) and including one or more morphological feature sets. The system may train the one or more machine learning models to identify physiological characteristics of users based on comparing the first set of morphological features and/or the second set of morphological features with the morphological feature sets of the training dataset.

For example, the system may identify one or more ground-truth labels associated with the training dataset. The one or more ground-truth labels may be physiological characteristics associated with the one or more users. The system may adjust one or more weights or parameters of the one or more machine learning models such that the one or more machine learning models may generate the one or more ground-truth labels when the system inputs the training dataset into the one or more machine learning models. Additionally, or alternatively, the system may correlate the physiological characteristics of the one or more users with related morphological feature sets of the training dataset. The one or more machine learning models may therefore be trained to output a first correlated physiological characteristic upon receiving an input including a set of data including morphological features that are within a threshold (e.g., a correlation threshold) of the correlated morphological feature set of the training dataset.

In some examples, the first set of morphological features associated with the PPG data (e.g., morphological characteristics of the time-domain representations 305) may include a correlation coefficient associated with subsets of the PPG data associated with different wavelengths, a time delay between systolic and diastolic peaks within the PPG data, a first derivative of the PPG data, and/or a second derivative of the PPG data. The second set of morphological features associated with the reconstructed PPG projections 310 may include a density of the reconstructed PPG projection 310 in the 3D space (e.g., how close/far apart the lines of the reconstructed PPG projections 310 are in 3D space), a width of a waveform of the reconstructed PPG projection 310 in the 3D space, a variability metric associated with the reconstructed PPG projection 310 in the 3D space (e.g., how similar the features of adjacent reconstructed projections are in 3D space), a correlation dimension metric, a Lyapunov exponent metric, an entropy metric, and/or an attractor geometry metric. In some examples, if the wearable device 304 collects multiple sets of PPG data (e.g., using multiple wavelengths of light), the system may identify the second set of morphological features based on comparing a first reconstructed PPG projection 310 associated with a first wavelength of light with a second reconstructed PPG projection 310 associated with a second wavelength of light.

To identify the physiological characteristics of the user, the system may compare the second set of morphological features to the morphological feature sets of the training dataset. The one or more machine learning models may identify and output the physiological characteristics of the user based on the comparison. For example, the one or more machine learning models may output a first correlated physiological characteristic upon receiving an input including a set of data including morphological features that are within a threshold (e.g., a correlation threshold) of the correlated morphological feature set of the training dataset.

In some examples, the system (e.g., the wearable device 304, the user device 106) may perform one or more additional physiological measurements based on the one or more physiological characteristics. For example, the system may determine a blood pressure metric, a blood oxygen saturation metric, a heart rate metric, a HRV metric, a cardiovascular age metric, and so on associated with the user based on identifying the physiological characteristics from the morphological features of the time-domain representation 305, the reconstructed PPG projections 310, or both.

As an illustrative example, FIG. 3 may illustrate example PPG data from three different users. Wearable devices 304 may collect the first time-domain representation 305-a of PPG data from a first user, the second time-domain representation 305-b of PPG data from a second user, and the third time-domain representation 305-c of PPG data from a third user. In other words, instead of illustrating different time-domain representations 305 associated with PPG data collected from a single user using different wavelengths, the time-domain representations may additionally, or alternatively, represent PPG data collected from different users in this example. In this example, the first user may be younger than the second user, and the second user may be younger than the third user. Further, the first user may exhibit the best cardiovascular health of the respective users, and the third user may exhibit the worst cardiovascular health of the respective users.

Continuing with the same example, the system may generate a reconstructed PPG projection 310 (e.g., a SPAR representation) of each time-domain representation 305 of the PPG data. For example, the system may generate a reconstructed PPG projection 310-a from the time-domain representation 305-a of the first user, a reconstructed PPG projection 310-b from the time-domain representation 305-b of the second user, and a reconstructed PPG projection 310-c from the time-domain representation 305-c of the third user. As illustrated with reference to FIG. 3, PPG data collected from the first user (e.g., with relatively better cardiovascular health) may be associated with a relatively less triangular-shaped reconstructed PPG projection 310-a (e.g., with relatively more defined “loops” in each corner of the reconstructed PPG projection 310-a). PPG data collected from the second user (e.g., with cardiovascular health that falls in between the first user and the third user) may be associated with a relatively more triangular-shaped reconstructed PPG projection 310-b than the reconstructed PPG projection 310-a (e.g., with relatively less defined “loops” in each corner of the reconstructed PPG projection 310-b). PPG data collected from the third user (e.g., with relatively worse cardiovascular health) may be associated with a relatively most triangular-shaped reconstructed PPG projection 310-c than the reconstructed PPG projection 310-a and the reconstructed PPG projection 310-b (e.g., without defined “loops” in each corner of the reconstructed PPG projection 310-c).

In some examples, the system may use a density 315 associated with each reconstructed PPG projection 310 to perform physiological measurements and/or to determine physiological characteristics or health-related insights for each user. For example, the reconstructed PPG projection 310-a may be associated with a relatively highest average density 315-a, which may indicate that the first user has a relatively better cardiovascular health (e.g., a relatively more regular blood pressure and heart rate, a relatively younger cardiovascular age). The reconstructed PPG projection 310-c may be associated with a relatively lowest average density 315-c, which may indicate that the third user has a relatively worse cardiovascular health (e.g., a relatively less regular blood pressure and heart rate, a relatively older cardiovascular age). The reconstructed PPG projection 310-b may be associated with an average density 315-b between the density 315-a and the density 315-c, which may indicate that the second user has a cardiovascular health that falls in between the cardiovascular health of the first user and the third user (e.g., a blood pressure and heart rate regularity that falls between the relatively better regularity and the relatively worse regularity, a cardiovascular age that falls between the relatively younger cardiovascular age and the relatively older cardiovascular age).

Accordingly, for the first user, the system may input the time-domain representation 305-a, the reconstructed PPG projection 310-a, and/or the density 315-a of the reconstructed PPG projection 310-a into the one or more machine learning models. The one or more machine learning models may, for example, identify that the first user has a relatively lower cardiovascular health and a relatively regular blood pressure and heart rate (e.g., by comparing the respective data/graphs to training datasets). Accordingly, the system may use the one or more machine learning models to identify the physiological characteristics of the first user. The system may perform a similar procedure to identify the related physiological characteristics of the second user and the third user. In other words, the system may be configured to identify a reconstructed PPG projection 310 of a user, and compare the reconstructed PPG projection 310 of other users that exhibit varying ages and/or varying cardiovascular health to evaluate the respective user's cardiovascular health and/or other physiological metrics.

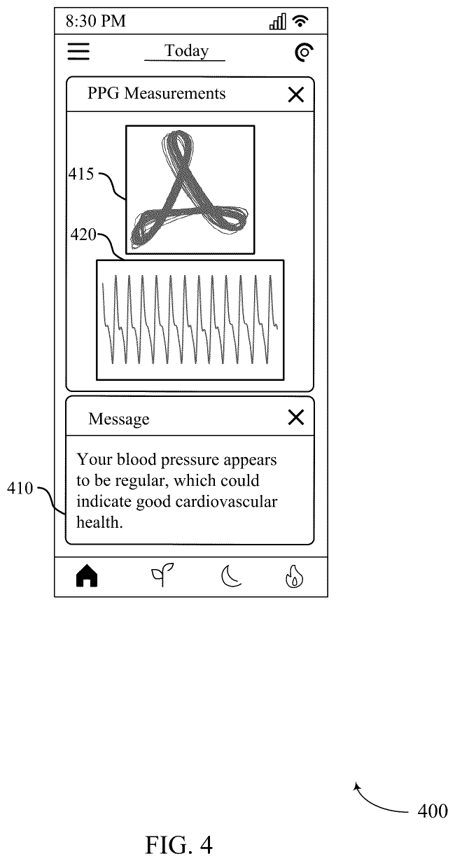

In some examples, as illustrated with reference to FIG. 4, the wearable device 304 (e.g., or the user device 106) may transmit one or more signals to cause a GUI of the user device 106 to display the PPG data. For example, the user device may display the time-domain representation 305, the reconstructed PPG projection 310, the density 315, and/or one or more messages related to cardiovascular health of the user (e.g., based on acquiring the additional physiological data). In some aspects, the reconstructed PPG projection 310 and/or the one or more messages may enable the user to better understand the PPG data collected from the user.

FIG. 4 shows an example of a GUI 400 that supports techniques for PPG analysis based on attractor reconstruction in accordance with aspects of the present disclosure. The GUI 400 may implement, or may be implemented by, aspects of the system 100, the system 200, and the data diagram 300. For example, the GUI 400 may be a GUI of a user device 106, which may be an example of a user device 106 as described with reference to FIG. 1.

In some examples, a wearable device 104 (e.g., a wearable ring device, a wrist-worn wearable device) may collect physiological data via one or more sensors (e.g., light-emitting components, light-receiving components). The physiological data may include, for example, a time-domain signal 420 of PPG data (e.g., a time-domain representation of a PPG signal of the user). The wearable device 104 may transmit the physiological data to a user device 106. In some examples, the wearable device 104, the user device 106, or both may perform an attractor reconstruction procedure to project the PPG data from the time-domain signal 420 to a reconstructed PPG projection 415 (e.g., a SPAR projection) as described with reference to FIG. 3. The user device (e.g., or the wearable device 104) may generate information related to the physiological measurements and/or perform additional physiological measurements based on the reconstructed PPG projection 415 (e.g., using one or more machine learning models).

The wearable device 104 (e.g., or the user device 106) may transmit one or more signals to cause a GUI 400 of the user device 106 to display visualizations of the PPG signals (e.g., the reconstructed PPG projection 415, the time-domain signal 420). In some examples, the reconstructed PPG projection 415 may be a 2D visualization of the reconstructed PPG projection in a 2D space and/or a 3D visualization of the reconstructed PPG projection in a 3D space. For example, as described with reference to FIG. 3, the GUI 400 may display the 3D representation of the PPG data and/or the 2D projection of the PPG data into the (v, w) plane.

In some cases, the visualization of the reconstructed PPG projection 415 described herein may provide benefits over traditional visualizations of time-domain signals 420. For example, the visualization of the reconstructed PPG projection 415 may be more intuitive for the user to understand, as the various heart beats of the user (represented by one “loop” around the reconstructed PPG projection 415) may be overlaid on top of one another, as opposed to separated in space as is the case with the time-domain signal 420. As such, by overlaying respective heart beats with the PPG signal on top of one another in the reconstructed PPG projection 415, the user may be able to more quickly and easily see and understand how different heart beats correlate with one another (e.g., how similar the heart beats are).

Moreover, in the context of a 3D projection, the user may be able to manipulate the reconstructed PPG projection 415 to see the visualization of the reconstructed PPG projection 415 from different perspectives in 3D space, which further improves the visualization of the reconstructed PPG projection 415 over conventional approaches for displaying PPG data. For example, the user may be able to manipulate the visualization of the reconstructed PPG projection 415 in order to view from a first perspective where the respective heart beats of the reconstructed PPG projection 415 are overlaid on top of one another (as shown in the reconstructed PPG projection 415 in FIG. 4), and may be able to further manipulate the visualization of the reconstructed PPG projection 415 in order to view from a second perspective where the respective heart beats of the reconstructed PPG projection 415 over time are separated in from one another spatially as a function of time (similar to conventional time-domain representations of PPG signals, where heart beats are separated spatially as a function of time). In this regard, the aspects of the present disclosure may enable more customizable and intuitive options for viewing and evaluating a user's PPG data using the visualization of the reconstructed PPG projection 415.

In some examples, the user device 106 may display an animation of the reconstructed PPG projection 415 and/or the time-domain signal 420 via the GUI 400. For example, the user device 106 may display an animation of the reconstructed PPG projection 415 and/or the time-domain signal 420 changing over time or an animation of the reconstructed PPG projection 415 rotating in 3D space. In some examples, the user device 106 may display a density of the reconstructed PPG projection 415, as illustrated with reference to FIG. 3.

The wearable device 104 (e.g., or the user device 106) may transmit one or more signals to cause a GUI 400 of the user device 106 to display one or more messages 410 including information associated with the physiological data. In some examples, the one or more messages 410 may include indications of the additional physiological data (e.g., a blood pressure metric, an SpO2 metric, a heart rate metric, a HRV metric, a cardiovascular age metric).

In some examples, the one or more messages 410 may include an explanation of the reconstructed PPG projection 415. For example, the one or more messages 410 may describe how the reconstructed PPG projection 415 relates to the heart health (e.g., cardiovascular age) of the user. As an illustrative example, the one or more messages may state that a high density of the reconstructed PPG projection 415 indicates that the user has relatively regular blood pressure and/or heart rate, which may indicate that the user has relatively better cardiovascular health. Additionally, or alternatively, the one or more messages may state that a low density or relatively more triangular shape of the reconstructed PPG projection 415 indicates that the user has relatively irregular blood pressure and/or heart rate, which may indicate that the user has relatively worse cardiovascular health.

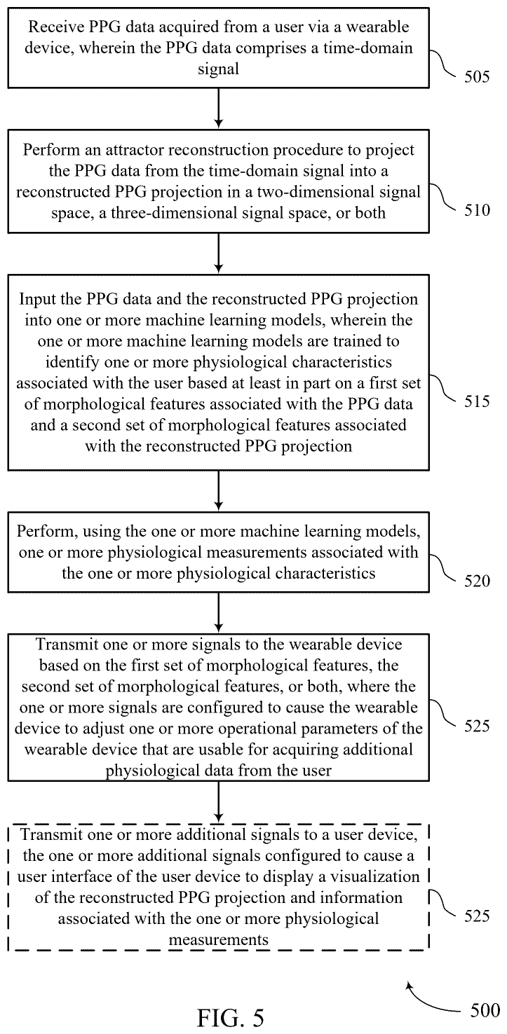

FIG. 5 shows a flowchart illustrating a method 500 that supports techniques for PPG analysis based on attractor reconstruction in accordance with aspects of the present disclosure. The operations of the method 500 may be implemented by a wearable device or its components as described herein. For example, the operations of the method 500 may be performed by a wearable device as described with reference to FIGS. 1 through 4. In some examples, a wearable device may execute a set of instructions to control the functional elements of the wearable device to perform the described functions. Additionally, or alternatively, the wearable device may perform aspects of the described functions using special-purpose hardware.

At 505, the method may include receiving PPG data acquired from a user via a wearable device, wherein the PPG data comprises a time-domain signal. The operations of 505 may be performed in accordance with examples as disclosed herein.

At 510, the method may include performing an attractor reconstruction procedure to project the PPG data from the time-domain signal into a reconstructed PPG projection in a 2D signal space, a 3D signal space, or both. The operations of 510 may be performed in accordance with examples as disclosed herein.

At 515, the method may include inputting the PPG data and the reconstructed PPG projection into one or more machine learning models, wherein the one or more machine learning models are trained to identify one or more physiological characteristics associated with the user based at least in part on a first set of morphological features associated with the PPG data and a second set of morphological features associated with the reconstructed PPG projection. The operations of 515 may be performed in accordance with examples as disclosed herein.

At 520, the method may include performing, using the one or more machine learning models, one or more physiological measurements associated with the one or more physiological characteristics. The operations of 520 may be performed in accordance with examples as disclosed herein.

At 525, the method may include transmitting one or more signals to the wearable device based on the first set of morphological features, the second set of morphological features, or both, where the one or more signals are configured to cause the wearable device to adjust one or more operational parameters (e.g., LED intensity, wavelengths of light transmitted by LEDs, etc.) of the wearable device that are usable for acquiring additional physiological data from the user. The operations of 525 may be performed in accordance with examples as disclosed herein.

At 530, the method may include transmitting one or more additional signals to a user device, the one or more additional signals configured to cause a user interface of the user device to display a visualization of the reconstructed PPG projection and information associated with the one or more physiological measurements. The operations of 530 may be performed in accordance with examples as disclosed herein.

It should be noted that the methods described above describe possible implementations, and that the operations and the steps may be rearranged or otherwise modified and that other implementations are possible. Furthermore, aspects from two or more of the methods may be combined.

A method by an apparatus is described. The method may include a wearable device configured to acquire physiological data from a user using one or more light-emitting components and one or more light-receiving components, the physiological data comprising at least PPG data from a user via a wearable device, a user device communicatively coupled with the wearable device, one or more processors communicatively coupled with the wearable device, the user device, or both, the one or more processors configured to, receive the PPG data acquired from the user via the wearable device, wherein the PPG data comprises a time-domain signal, perform an attractor reconstruction procedure to project the PPG data from the time-domain signal into a reconstructed PPG projection in a 2D signal space, a 3D signal space, or both, input the PPG data and the reconstructed PPG projection into one or more machine learning models, wherein the one or more machine learning models are trained to identify one or more physiological characteristics associated with the user based at least in part on a first set of morphological features associated with the PPG data and a second set of morphological features associated with the reconstructed PPG projection, perform, using the one or more machine learning models, one or more physiological measurements associated with the one or more physiological characteristics, and transmit one or more signals to the user device, the one or more signals configured to cause a user interface of the user device to display a visualization of the reconstructed PPG projection and information associated with the one or more physiological measurements.

An apparatus is described. The apparatus may include one or more memories storing processor executable code, and one or more processors coupled with the one or more memories. The one or more processors may individually or collectively be operable to execute the code to cause the apparatus to a wearable device configure to acquire physiological data from a user using one or more light-emitting components and one or more light-receiving components, the physiological data comprising at least PPG data from a user via a wearable device, a user device communicatively couple with the wearable device, one or more processors communicatively couple with the wearable device, the user device, or both, the one or more processors configured to, receive the PPG data acquired from the user via the wearable device, wherein the PPG data comprises a time-domain signal, perform an attractor reconstruction procedure to project the PPG data from the time-domain signal into a reconstructed PPG projection in a 2D signal space, a 3D signal space, or both, input the PPG data and the reconstructed PPG projection into one or more machine learning models, wherein the one or more machine learning models are trained to identify one or more physiological characteristics associated with the user based at least in part on a first set of morphological features associated with the PPG data and a second set of morphological features associated with the reconstructed PPG projection, perform, using the one or more machine learning models, one or more physiological measurements associated with the one or more physiological characteristics, and transmit one or more signals to the user device, the one or more signals configured to cause a user interface of the user device to display a visualization of the reconstructed PPG projection and information associated with the one or more physiological measurements.

Another apparatus is described. The apparatus may include means for a wearable device configured to acquire physiological data from a user using one or more light-emitting components and one or more light-receiving components, the physiological data comprising at least PPG data from a user via a wearable device, means for a user device communicatively coupled with the wearable device, means for one or more processors communicatively coupled with the wearable device, the user device, or both, the one or more processors configured to, means for receive the PPG data acquired from the user via the wearable device, wherein the PPG data comprises a time-domain signal, means for perform an attractor reconstruction procedure to project the PPG data from the time-domain signal into a reconstructed PPG projection in a 2D signal space, a 3D signal space, or both, means for input the PPG data and the reconstructed PPG projection into one or more machine learning models, wherein the one or more machine learning models are trained to identify one or more physiological characteristics associated with the user based at least in part on a first set of morphological features associated with the PPG data and a second set of morphological features associated with the reconstructed PPG projection, means for perform, using the one or more machine learning models, one or more physiological measurements associated with the one or more physiological characteristics, and means for transmit one or more signals to the user device, the one or more signals configured to cause a user interface of the user device to display a visualization of the reconstructed PPG projection and information associated with the one or more physiological measurements.

A non-transitory computer-readable medium storing code is described. The code may include instructions executable by one or more processors to a wearable device configure to acquire physiological data from a user using one or more light-emitting components and one or more light-receiving components, the physiological data comprising at least PPG data from a user via a wearable device, a user device communicatively couple with the wearable device, one or more processors communicatively couple with the wearable device, the user device, or both, the one or more processors configured to, receive the PPG data acquired from the user via the wearable device, wherein the PPG data comprises a time-domain signal, perform an attractor reconstruction procedure to project the PPG data from the time-domain signal into a reconstructed PPG projection in a 2D signal space, a 3D signal space, or both, input the PPG data and the reconstructed PPG projection into one or more machine learning models, wherein the one or more machine learning models are trained to identify one or more physiological characteristics associated with the user based at least in part on a first set of morphological features associated with the PPG data and a second set of morphological features associated with the reconstructed PPG projection, perform, using the one or more machine learning models, one or more physiological measurements associated with the one or more physiological characteristics, and transmit one or more signals to the user device, the one or more signals configured to cause a user interface of the user device to display a visualization of the reconstructed PPG projection and information associated with the one or more physiological measurements.

In some examples of the method, apparatus, and non-transitory computer-readable medium described herein, the second set of morphological features associated with the reconstructed PPG projection comprise a density of the reconstructed PPG projection in the 3D signal space, a width of a waveform of the reconstructed PPG projection in the 3D signal space, a variability metric associated with the reconstructed PPG projection in the 3D signal space, a correlation dimension metric, a Lyapunov exponent metric, an entropy metric, an attractor geometry metric, or any combination thereof.

In some examples of the method, apparatus, and non-transitory computer-readable medium described herein, and the method, apparatuses, and non-transitory computer-readable medium may include further operations, features, means, or instructions for perform an additional attractor reconstruction procedure to project the additional time-domain signal associated with the second wavelength into an additional reconstructed PPG projection in the 2D signal space, the 3D signal space, or both and input the PPG data, the reconstructed PPG projection, and the additional reconstructed PPG projection into the one or more machine learning models, wherein the one or more machine learning models may be trained to identify the one or more physiological characteristics associated with the user based at least in part on the first set of morphological features associated with the PPG data, the second set of morphological features associated with the reconstructed PPG projection, and a third set of morphological features associated with the additional reconstructed PPG projection.

In some examples of the method, apparatus, and non-transitory computer-readable medium described herein, the second set of morphological features associated with the reconstructed PPG projection may be based at least in part on a comparison between the reconstructed PPG projection associated with the first wavelength and the additional reconstructed PPG projection associated with the second wavelength in the 3D signal space.

Some examples of the method, apparatus, and non-transitory computer-readable medium described herein may further include operations, features, means, or instructions for determine a data quality metric associated with the PPG data, wherein the attractor reconstruction procedure may be performed based at least in part on the data quality metric satisfying a threshold quality metric.

Some examples of the method, apparatus, and non-transitory computer-readable medium described herein may further include operations, features, means, or instructions for compare the second set of morphological features to a plurality of morphological feature sets associated with a plurality of additional users associated with a plurality of demographic characteristics, wherein the one or more machine learning models may be configured to identify the one or more physiological characteristics of the user based at least in part on the comparison between the second set of morphological features and the plurality of morphological feature sets.

In some examples of the method, apparatus, and non-transitory computer-readable medium described herein, the first set of morphological features associated with the PPG data comprise a correlation coefficient associated with subsets of the PPG data associated with different wavelengths, a time delay between systolic and diastolic peaks within the PPG data, a first derivative of the PPG data, a second derivative of the PPG data, or any combination thereof.

In some examples of the method, apparatus, and non-transitory computer-readable medium described herein, the one or more physiological measurements associated with the user comprise a blood pressure metric, a blood oxygen saturation metric, a heart rate metric, a HRV metric, a cardiovascular age metric, or any combination thereof.

In some examples of the method, apparatus, and non-transitory computer-readable medium described herein, the visualization of the reconstructed PPG projection comprises a 2D visualization of the reconstructed PPG projection in the 2D signal space, a 3D visualization of the reconstructed PPG projection in the 3D signal space, or both.

In some examples of the method, apparatus, and non-transitory computer-readable medium described herein, the attractor reconstruction procedure comprises a SPAR procedure.

In some examples of the method, apparatus, and non-transitory computer-readable medium described herein, the wearable device comprises a wearable ring device.

In some examples of the method, apparatus, and non-transitory computer-readable medium described herein, the wearable device comprises a wrist-worn wearable device.

A method for PPG measurement by an apparatus is described. The method may include receiving PPG data acquired from a user via a wearable device, wherein the PPG data comprises a time-domain signal, performing an attractor reconstruction procedure to project the PPG data from the time-domain signal into a reconstructed PPG projection in a 2D signal space, a 3D signal space, or both, inputting the PPG data and the reconstructed PPG projection into one or more machine learning models, wherein the one or more machine learning models are trained to identify one or more physiological characteristics associated with the user based at least in part on a first set of morphological features associated with the PPG data and a second set of morphological features associated with the reconstructed PPG projection, performing, using the one or more machine learning models, one or more physiological measurements associated with the one or more physiological characteristics, and transmitting one or more signals to a user device, the one or more signals configured to cause a user interface of the user device to display a visualization of the reconstructed PPG projection and information associated with the one or more physiological measurements.

An apparatus for PPG measurement is described. The apparatus may include one or more memories storing processor executable code, and one or more processors coupled with the one or more memories. The one or more processors may individually or collectively be operable to execute the code to cause the apparatus to receive PPG data acquired from a user via a wearable device, wherein the PPG data comprises a time-domain signal, perform an attractor reconstruction procedure to project the PPG data from the time-domain signal into a reconstructed PPG projection in a 2D signal space, a 3D signal space, or both, input the PPG data and the reconstructed PPG projection into one or more machine learning models, wherein the one or more machine learning models are trained to identify one or more physiological characteristics associated with the user based at least in part on a first set of morphological features associated with the PPG data and a second set of morphological features associated with the reconstructed PPG projection, perform, using the one or more machine learning models, one or more physiological measurements associated with the one or more physiological characteristics, and transmit one or more signals to a user device, the one or more signals configured to cause a user interface of the user device to display a visualization of the reconstructed PPG projection and information associated with the one or more physiological measurements.

Another apparatus for PPG measurement is described. The apparatus may include means for receiving PPG data acquired from a user via a wearable device, wherein the PPG data comprises a time-domain signal, means for performing an attractor reconstruction procedure to project the PPG data from the time-domain signal into a reconstructed PPG projection in a 2D signal space, a 3D signal space, or both, means for inputting the PPG data and the reconstructed PPG projection into one or more machine learning models, wherein the one or more machine learning models are trained to identify one or more physiological characteristics associated with the user based at least in part on a first set of morphological features associated with the PPG data and a second set of morphological features associated with the reconstructed PPG projection, means for performing, using the one or more machine learning models, one or more physiological measurements associated with the one or more physiological characteristics, and means for transmitting one or more signals to a user device, the one or more signals configured to cause a user interface of the user device to display a visualization of the reconstructed PPG projection and information associated with the one or more physiological measurements.

A non-transitory computer-readable medium storing code for PPG measurement is described. The code may include instructions executable by one or more processors to receive PPG data acquired from a user via a wearable device, wherein the PPG data comprises a time-domain signal, perform an attractor reconstruction procedure to project the PPG data from the time-domain signal into a reconstructed PPG projection in a 2D signal space, a 3D signal space, or both, input the PPG data and the reconstructed PPG projection into one or more machine learning models, wherein the one or more machine learning models are trained to identify one or more physiological characteristics associated with the user based at least in part on a first set of morphological features associated with the PPG data and a second set of morphological features associated with the reconstructed PPG projection, perform, using the one or more machine learning models, one or more physiological measurements associated with the one or more physiological characteristics, and transmit one or more signals to a user device, the one or more signals configured to cause a user interface of the user device to display a visualization of the reconstructed PPG projection and information associated with the one or more physiological measurements.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A system for photoplethysmogram (PPG) measurement, comprising:
a wearable device configured to acquire physiological data from a user using one or more light-emitting components and one or more light-receiving components, the physiological data comprising at least PPG data;
a user device communicatively coupled with the wearable device; and
one or more processors communicatively coupled with the wearable device, the user device, or both, the one or more processors configured to:
receive the PPG data acquired from the user via the wearable device, wherein the PPG data comprises a time-domain signal;
perform an attractor reconstruction procedure to project the PPG data from the time-domain signal into a reconstructed PPG projection in a two-dimensional signal space, a three-dimensional signal space, or both;
input the PPG data and the reconstructed PPG projection into one or more machine learning models, wherein the one or more machine learning models are trained to identify one or more physiological characteristics associated with the user based at least in part on a first set of morphological features associated with the PPG data and a second set of morphological features associated with the reconstructed PPG projection;
perform, using the one or more machine learning models, one or more physiological measurements associated with the one or more physiological characteristics; and
transmit one or more signals to the wearable device based at least in part on the first set of morphological features, the second set of morphological features, or both, wherein the one or more signals are configured to cause the wearable device to adjust one or more operational parameters of the wearable device that are usable for acquiring additional physiological data from the user.

2. The system of claim 1, wherein the second set of morphological features associated with the reconstructed PPG projection comprise a density of the reconstructed PPG projection in the three-dimensional signal space, a width of a waveform of the reconstructed PPG projection in the three-dimensional signal space, a variability metric associated with the reconstructed PPG projection in the three-dimensional signal space, a correlation dimension metric, a Lyapunov exponent metric, an entropy metric, an attractor geometry metric, or any combination thereof.

3. The system of claim 1, wherein the wearable device is configured to collect the physiological data using light associated with at least a first wavelength and a second wavelength, wherein the PPG data comprises at least the time-domain signal associated with the first wavelength, and an additional time-domain signal associated with the second wavelength, wherein the one or more processors are further configured to:
perform an additional attractor reconstruction procedure to project the additional time-domain signal associated with the second wavelength into an additional reconstructed PPG projection in the two-dimensional signal space, the three-dimensional signal space, or both; and
input the PPG data, the reconstructed PPG projection, and the additional reconstructed PPG projection into the one or more machine learning models, wherein the one or more machine learning models are trained to identify the one or more physiological characteristics associated with the user based at least in part on the first set of morphological features associated with the PPG data, the second set of morphological features associated with the reconstructed PPG projection, and a third set of morphological features associated with the additional reconstructed PPG projection.

4. The system of claim 3, wherein the second set of morphological features associated with the reconstructed PPG projection is based at least in part on a comparison between the reconstructed PPG projection associated with the first wavelength and the additional reconstructed PPG projection associated with the second wavelength in the three-dimensional signal space.

5. The system of claim 1, wherein the one or more processors are further configured to:
determine a data quality metric associated with the PPG data, wherein the attractor reconstruction procedure is performed based at least in part on the data quality metric satisfying a threshold quality metric.

6. The system of claim 1, wherein the one or more processors are further configured to:
compare the second set of morphological features to a plurality of morphological feature sets associated with a plurality of additional users associated with a plurality of demographic characteristics, wherein the one or more machine learning models are configured to identify the one or more physiological characteristics of the user based at least in part on the comparison between the second set of morphological features and the plurality of morphological feature sets.

7. The system of claim 1, wherein the first set of morphological features associated with the PPG data comprise a correlation coefficient associated with subsets of the PPG data associated with different wavelengths, a time delay between systolic and diastolic peaks within the PPG data, a first derivative of the PPG data, a second derivative of the PPG data, or any combination thereof.

8. The system of claim 1, wherein the one or more physiological measurements associated with the user comprise a blood pressure metric, a blood oxygen saturation metric, a heart rate metric, a heart rate variability metric, a cardiovascular age metric, or any combination thereof.

9. The system of claim 1, wherein the one or more processors are further configured to:
transmit one or more additional signals to the user device, the one or more signals configured to cause a user interface of the user device to display a visualization of the reconstructed PPG projection and information associated with the one or more physiological measurements.

10. The system of claim 9, wherein the visualization of the reconstructed PPG projection comprises a two-dimensional visualization of the reconstructed PPG projection in the two-dimensional signal space, a three-dimensional visualization of the reconstructed PPG projection in the three-dimensional signal space, or both.

11. The system of claim 1, wherein the attractor reconstruction procedure comprises a symmetric projection attractor reconstruction (SPAR) procedure.

12. The system of claim 1, wherein the wearable device comprises a wearable ring device configured to be worn on a finger of the user.

13. The system of claim 1, wherein the wearable device comprises a wrist-worn wearable device.

14. A method for photoplethysmogram (PPG) measurement, comprising:
acquiring physiological data from a user using one or more light-emitting components and one or more light-receiving components of a wearable device, the physiological data comprising at least PPG data, wherein the PPG data comprises a time-domain signal;
performing an attractor reconstruction procedure to project the PPG data from the time-domain signal into a reconstructed PPG projection in a two-dimensional signal space, a three-dimensional signal space, or both;
inputting the PPG data and the reconstructed PPG projection into one or more machine learning models, wherein the one or more machine learning models are trained to identify one or more physiological characteristics associated with the user based at least in part on a first set of morphological features associated with the PPG data and a second set of morphological features associated with the reconstructed PPG projection;
performing, using the one or more machine learning models, one or more physiological measurements associated with the one or more physiological characteristics; and
adjusting one or more operational parameters of the wearable device that are usable by the wearable device for acquiring additional physiological data from the user based at least in part on the first set of morphological features, the second set of morphological features, or both.

15. The method of claim 14, wherein the second set of morphological features associated with the reconstructed PPG projection comprise a density of the reconstructed PPG projection in the three-dimensional signal space, a width of a waveform of the reconstructed PPG projection in the three-dimensional signal space, a variability metric associated with the reconstructed PPG projection in the three-dimensional signal space, a correlation dimension metric, a Lyapunov exponent metric, an entropy metric, an attractor geometry metric, or any combination thereof.

16. The method of claim 14, wherein the wearable device is configured to collect the physiological data using light associated with at least a first wavelength and a second wavelength, wherein the PPG data comprises at least the time-domain signal associated with the first wavelength, and an additional time-domain signal associated with the second wavelength, the method further comprising:
performing an additional attractor reconstruction procedure to project the additional time-domain signal associated with the second wavelength into an additional reconstructed PPG projection in the two-dimensional signal space, the three-dimensional signal space, or both; and
inputting the PPG data, the reconstructed PPG projection, and the additional reconstructed PPG projection into the one or more machine learning models, wherein the one or more machine learning models are trained to identify the one or more physiological characteristics associated with the user based at least in part on the first set of morphological features associated with the PPG data, the second set of morphological features associated with the reconstructed PPG projection, and a third set of morphological features associated with the additional reconstructed PPG projection.

17. The method of claim 16, wherein the second set of morphological features associated with the reconstructed PPG projection is based at least in part on a comparison between the reconstructed PPG projection associated with the first wavelength and the additional reconstructed PPG projection associated with the second wavelength in the three-dimensional signal space.

18. The method of claim 14, further comprising:
determining a data quality metric associated with the PPG data, wherein the attractor reconstruction procedure is performed based at least in part on the data quality metric satisfying a threshold quality metric.

19. The method of claim 14, further comprising:
comparing the second set of morphological features to a plurality of morphological feature sets associated with a plurality of additional users associated with a plurality of demographic characteristics, wherein the one or more machine learning models are configured to identify the one or more physiological characteristics of the user based at least in part on the comparison between the second set of morphological features and the plurality of morphological feature sets.

20. A method for photoplethysmogram (PPG) measurement, comprising:
acquiring physiological data from a user using one or more light-emitting components and one or more light-receiving components of a wearable device, the physiological data comprising at least PPG data, wherein the PPG data comprises a time-domain signal;
performing an attractor reconstruction procedure to project the PPG data from the time-domain signal into a reconstructed PPG projection in a two-dimensional signal space, a three-dimensional signal space, or both;
inputting the PPG data and the reconstructed PPG projection into one or more machine learning models, wherein the one or more machine learning models are trained to identify one or more physiological characteristics associated with the user based at least in part on a first set of morphological features associated with the PPG data and a second set of morphological features associated with the reconstructed PPG projection;
performing, using the one or more machine learning models, one or more physiological measurements associated with the one or more physiological characteristics; and
transmit one or more signals to a user device, the one or more signals configured to cause a user interface of the user device to display a visualization of the reconstructed PPG projection and information associated with the one or more physiological measurements.
